(19) United States
(12) Patent Application Publication
(10) Pub. No.: US 2004/0260119 A1
(43) Pub. Date: Dec. 23, 2004

(54) Preparation of 3-alkoxy-2-methylbenzoic acids

(76) Inventors: Johannes Scherer, Leverkusen (DE); Friedrich Mueller-Hauck, Bendorf (DE)

Correspondence Address: LANXESS CORPORATION, PATENT DEPARTMENT/ BLDG 14, 100 BAYER ROAD, PITTSBURGH, PA, 15205-9741, US

(21) Appl. No.: 10/458,057

(22) Filed: Jun. 10, 2003

(30) Foreign Application Priority Data
Jun. 13, 2002 (DE) 10226218.7

(51) Int. Cl. C07C 229/38

(52) U.S. Cl. 562/452; 562/473

(57) ABSTRACT

The invention relates to an improved process for preparing 3-alkoxy-2-methylbenzoic acids by heating substituted naphthalenes in the presence of alkali metal hydroxides and subsequently alkylating.

### BACKGROUND OF THE INVENTION

1. Field of the Invention

The invention relates to an improved process for preparing 3-alkoxy-2-methylbenzoic acids by heating substituted naphthalenes in the presence of alkali metal hydroxides and subsequently alkylating.

2. Brief Description of the Prior Art

3-Alkoxy-2-methylbenzoic acids, for example 3-methoxy-2-methylbenzoic acid, are valuable intermediates in the preparation of pharmaceuticals and agrochemicals, for example insecticides (see, for example, U.S. Pat. No. 5,484,926 and EP-A 639 559).

According to Dean et al. (J. Chem. Soc., 1961, 2773), 3-methoxy-2-methylbenzoic acid can be prepared by heating sodium hydrogen 3-aminonaphthalene-1,5-disulphonic acid with two equivalents by weight of sodium hydroxide and water to from 275 to 280° C. at a nitrogen pressure of 40 bar and, after cooling the reaction mixture, initially obtaining the 3-hydroxy-2-methylbenzoic acid by filtration and acidification, and subsequently reacting it with dimethyl sulphate in a manner not described in detail.

However, the method has the disadvantage that large amounts of alkalis have to be used and result in a large amount of waste salts in the workup. The 3-hydroxy-2-methylbenzoic acid occurring as an intermediate also initially has to be isolated before the further reaction.

There is therefore a need to develop an efficient process which enables the preparation of 3-alkoxy-2-methylbenzoic acid in an advantageous manner.

### SUMMARY OF THE INVENTION

A process has now been found for preparing compounds of the formula (I)

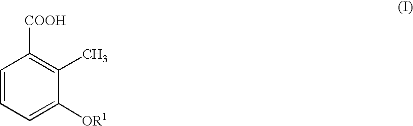

where

R¹ is C₁-C₁₄-alkyl, C₇-C₂₀-arylalkyl, C₁₃-C₂₀-diarylalkyl or radicals of the formulae (IIa) or (IIb)

A—OR² (IIa)

A—NR³R⁴ (IIb)

where A is in each case a C₁-C₄-alkylene radical and R² and also R³ and R⁴ are each independently methyl, ethyl and isopropyl,

which is characterized in that

a) compounds of the formula (III)

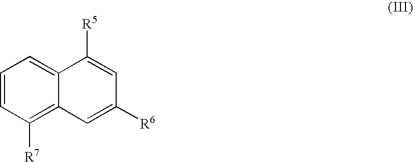

where

R⁵, R⁶ and R⁷ are each independently hydrogen, hydroxyl, amino or SO₃M where M is hydrogen, ammonium, an alkali metal or half an equivalent of an alkaline earth metal,

are reacted with alkali metal hydroxide and optionally alkaline earth metal hydroxide in the presence of water and

b) the reaction mixtures (obtained in step a),

optionally after addition of water and

optionally after removal of insoluble constituents and

optionally after the separation of undesired, soluble constituents,

are partially neutralized and

c) the reaction mixtures obtained in step b) are reacted with compounds of the formulae (IVa), (IVb) or (IVc)

R¹—X (IVa)

R¹—OSO₂—R⁸ (IVb)

R¹—OSO₂—OR¹ (IVc)

where R¹ is as defined above and

X is chlorine, bromine or iodine and

R⁸ is C₁-C₄-alkyl, C₁-C₄-perfluoroalkyl, phenyl or p-tosyl and

d) the reaction mixtures obtained in step c) are acidified.

For the purposes of the invention, alkyl and alkylene are each independently a straight-chain, cyclic, branched or unbranched alkyl and alkylene radical respectively. The same applies to the alkylene moiety of an aralkyl radical.

### DETAILED DESCRIPTION OF THE INVENTION

In all contexts, C₁-C₁₄-alkyl is, for example, preferably methyl, ethyl, n-propyl, isopropyl, n-butyl, tert-butyl, n-pentyl, cyclohexyl, n-hexyl, n-heptyl, n-octyl, isooctyl, n-decyl and n-dodecyl.

In all contexts, C₁-C₄-alkylene is, for example, preferably methylene, 1,1-ethylene, 1,2-ethylene, 1,1-propylene, 1,2-propylene, 1,3-propylene, 1,1-butylene, 1,2-butylene, 2,3-butylene and 1,4-butylene.

For the purposes of the invention, arylalkyl is, for example and with preference, an alkyl radical which is substituted as defined above by carbocyclic aromatic radicals having 6 to 10 carbon atoms, in particular phenyl or naphthyl, and the carbocyclic aromatic radicals may themselves be substituted by up to five substituents per cycle which are selected from the group of methyl, ethyl, fluorine, chlorine, bromine and C₁-C₄-fluoroalkyl where fluoroalkyl is an alkyl radical as defined above which is singly, multiply or fully substituted by fluorine. Preferred C₁-C₄-fluoroalkyl radicals are trifluoromethyl, pentafluoroethyl, 2,2,2-trifluoroethyl and nonafluorobutyl. The same applies to the aryl moiety of a diarylalkyl radical.

Particular preference is given to using 3-amino-1,5-naphthalenedisulphonic acid and its mono- or dialkali metal salts, and also 1,3,5-naphthalenetrisulphonic acid and its mono-, di- or trialkali metal salts for step a). It is also possible to use mixtures of these compounds.

Very particular preference is given to using the trialkali metal salts of 1,3,5-naphthalenesulphonic acid, for example trisodium 1,3,5-naphthalenetrisulphonate and tripotassium 1,3,5-naphthalenetrisulphonate, and even greater preference is given to trisodium 1,3,5-naphthalenetrisulphonate.

Some of the compounds of the formula (II) can occur in the form of hydrates which are not mentioned specifically, but are encompassed by the invention.

The compounds of the formula (II) are either commercially available, or can be prepared by literature procedures.

In a preferred embodiment of the process according to the invention, the naphthalene 1,3,5-trisulphonic acid or its mono-, di- or trialkali metal salts are prepared in such a way that

i) naphthalene is reacted with fuming sulphuric acid to give naphthalene-1,3,5-trisulphonic acid and

ii) the naphthalene-1,3,5-trisulphonic acid obtained in step i) is optionally converted to a mono-, di- or trialkali metal salt.

At this point, it is pointed out that any desired combination of the features and of the areas of preference specified are likewise encompassed by the invention.

Step i) can advantageously be effected by reacting naphthalene with fuming sulphuric acid.

An example of a possible procedure is to initially charge fuming sulphuric acid and add naphthalene or initially charge concentrated sulphuric acid and naphthalene and add fuming sulphuric acid, or initially charge concentrated sulphuric acid and add naphthalene and fuming sulphuric acid.

The preferred procedure in step i) is to initially charge concentrated sulphuric acid and add naphthalene and fuming sulphuric acid.

For the purposes of the invention, concentrated sulphuric acid is, for example, sulphuric acid comprising 90 to 100% by weight of H₂SO₄. For the purposes of the invention, fuming sulphuric acid is sulphuric acid which has a content of over 100% by weight, based on pure H₂SO₄. Another common term for fuming sulphuric acid for the purposes of the invention is oleum.

Typically, the content of free SO₃ in commercially available oleum is specified and is, for example, 30 or 65% by weight.

Preference is given to using such an amount of oleum in step a) that the molar ratio of free SO₃ to naphthalene is between 1.5:1 and 10:1, preferably between 2:1 and 5:1 and more preferably between 2.5:1 and 4:1.

The temperature in the course of addition can be, for example, −20 to 70° C., preferably 20 to 55° C.

The time for the addition can be, for example, between 10 min and 48 h, preferably 2 to 24 hours.

Subsequently, the resulting reaction mixture can optionally be heated. The temperature can be, for example, between 55 and 150° C., preferably between 80 and 100° C.

The naphthalene 1,3,5-trisulphonic acid can be recovered from the resulting reaction mixture, for example, by adding water.

Alkali metal or alkaline earth metal salts of naphthalene-1,3,5-trisulphonic acid can be prepared according to step ii) either from the isolated naphthalene-1,3,5-trisulphonic acid or directly from the reaction mixture resulting from step i). Preference is given to the preparation of alkali metal or alkaline earth metal salts of naphthalene-1,3,5-trisulphonic acid from the reaction mixture resulting from step i).

Step ii) can be effected, for example, in such a way that the reaction mixture resulting from step i) is diluted, for example, by pouring into water or onto ice, and subsequently reacted with alkali metal hydroxides, hydrogencarbonates or carbonates or aqueous solutions thereof.

Preference is given to using alkali metal hydroxides, in particular sodium hydroxide and potassium hydroxide, or aqueous solutions thereof.

Particular preference is given to the reaction with aqueous solutions of sodium hydroxide.

The alkali metal hydroxide content of the solutions can be, for example, between 2 and 75% by weight, preferably from 25 to 60% by weight.

The temperature of the reaction for step ii) can be, for example, 0 to 100° C., preferably 80 to 100° C.

The amount of alkali metal hydroxide used can be, for example, 2 to 10 times, based on the molar ratio of the naphthalene used in step i), preferably 2.8 to 3.5 times.

After workup in a manner known per se, which can be effected, for example, by filtration and optionally washing and drying the precipitated solid, alkali metal salts of naphthalene-1,3,5-trisulphonic acid are obtained which are either stored or preferably reacted further.

Optionally, the alkali metal salts of naphthalene-1,3,5-trisulphonic acid obtained in step ii) may be still further purified, for example, by recrystallization, although this is unnecessary for use in step a) of the process according to the invention.

In step a) of the process according to the invention, the compounds of the formula (II) are reacted with alkali metal hydroxide and optionally alkaline earth metal hydroxide in the presence of water.

The alkali metal hydroxide used may be, for example and with preference, sodium hydroxide or potassium hydroxide or a mixture thereof, for example as a solid or in the form of an aqueous solution.

The amount of alkali metal hydroxide for step a) may be selected, for example, in such a way that, for each R¹ radical in the compounds of the formula (III) that is amino or SO₃M, 2 to 30 mol, preferably 3 to 10 mol, but a total of at least 2 mol, of alkali metal hydroxide are used per mole of the compound of the formula (II). When M is hydrogen, the amount of alkali metal hydroxide advantageously has to be increased in accordance with the molarity.

Alkaline earth metal hydroxides can preferably also be added. Examples of suitable alkaline earth metal hydroxides are magnesium hydroxide and calcium hydroxide, although preference is given to calcium hydroxide.

The amount of alkaline earth metal hydroxide for step a) may, for example, be selected in such a way that, for each R¹ radical in the compounds of the formula (III) that is amino or SO₃M, 0.5 to 20 mol, preferably 1 to 20 mol and more preferably 1.5 to 7 mol, but a total of at least 1 mol, of alkaline earth metal hydroxide are used per mole of the compound of the formula (II).

The molar ratio of water to the compound of the formula (III) can be 0.5 to 200 mol, preferably 3 to 50 mol.

In a particularly preferred embodiment, the ratio of water to the sum of alkali metal hydroxide and any alkaline earth metal hydroxide added is 1:1.4 to 1:6.0.

The pressure in the reaction can be, for example, 1 to 200 bar, preferably 1 to 100 bar and most preferably that pressure which results from heating the reaction mixture to the reaction temperature in a closed vessel starting from ambient temperature.

An example of a useful closed vessel is an autoclave which can be made, for example, of nickel, nickel-based alloys, silver or other, alkali-resistant material.

The temperature of the reaction can be, for example, 240 to 350° C., preferably 270 to 320° C.

The reaction time can be, for example, 2 to 25 hours, preferably 3 to 8 hours.

In step b) of the process according to the invention, the reaction mixtures obtained in step a) are at least partially neutralized with acid, optionally after adding water and optionally after the removal of insoluble constituents and optionally the removal of undesired, soluble constituents.

In a preferred embodiment, water is optionally added after cooling the reaction mixture, which can be effected, for example, by pouring the reaction mixture into water or onto ice.

Preference is also given to removing insoluble constituents. This may be effected, for example and with preference, by filtration, centrifugation, sedimentation and decanting, optionally in the presence of assistants. Examples of possible assistants include kieselguhr, for example Celite (1, activated carbon, for example Norite®, bleaching earth, montmorillonite or animal charcoal. Preference is given to filtration, particular preference to filtration in the presence of assistants, preferably activated carbon.

Preference is likewise given to the removal of undesired, soluble constituents. Undesired, soluble constituents are, for example, coloured organic by-products. The removal of undesired, soluble constituents may be carried out, for example, before or after the partial neutralization. An example of a possible procedure is to extract with organic solvent. Examples of suitable organic solvents are esters such as ethyl acetate and butyl acetate, aliphatic or aromatic, optionally halogenated hydrocarbons, for example petroleum, benzene, toluene, xylenes, chlorobenzene, dichlorobenzenes, isopropylbenzene, petroleum ether, hexane, heptane, octane, isooctane, cyclohexane, methylcyclohexane, dichloromethane, chloroform or carbon tetrachloride; ethers such as diethyl ether, methyl tert-butyl ether or diisopropyl ether, ketones such as 2-butanone or methyl isobutyl ketone or mixtures of such solvents.

The reaction mixture may also, for example, be freed of discolorations using a suitable adsorbent. Examples of suitable adsorbents include silica gels, aluminium oxides, cellulose or activated carbon.

The partial neutralization is preferably effected by setting to a pH of 8 to 13, preferably 9.5 to 11.5. The pH values relate to values at 25° C. For partial neutralization, preference is given to using acids or acidic salts having a pKa in water of 5 or less. For partial neutralization, preference is given to using sulphuric acid, hydrochloric acid, phosphoric acid, nitric acid and hydrobromic acid, particular preference is given to using sulphuric acid and hydrochloric acid, and very particular preference to using sulphuric acid.

In a very particularly preferred embodiment, step b) is carried out in such a way that the reaction mixture obtained from step a) is initially diluted with water, insolubles are removed, the pH is set to 8 to 13 and undesired, soluble constituents are removed.

In step c), the reaction mixture from step b) is reacted with compounds of the formulae (IVa), (IVb) or (IVc).

In the compounds of the formulae (IVa), (IVb) and (IVc), R¹ is preferably methyl, ethyl, isopropyl or benzyl, particularly preferably methyl.

Preferred compounds of the formula (IVa) are methyl chloride and methyl iodide, preferred compounds of the formula (IVb) are methyl mesylate and methyl p-tosylate, and the preferred compound of formula (IVc) which is most preferred for the process according to the invention is dimethyl sulphate.

The amount of compound of the formula (IVa), (IVb) or (IVc) is, for example and with preference, selected in such a way that the molar ratio to the compound of the formula (III) originally used is 1:1 to 10:1, preferably 2:1 to 7:1.

The reaction temperature in step c) is, for example, 0 to 120° C., preferably 20 to 100° C. When using compounds of the formulae (IVa), (IVb) and (IVc) which are gaseous at room temperature, such as methyl chloride in particular, the reaction may advantageously be carried out under pressure.

Ambient pressure is otherwise preferred, although the reaction pressure is not critical.

The reaction time for step c) can be, for example, 1 to 25 hours, preferably 2 to 10 hours.

Preference is further given to maintaining the pH in the course of the reaction between 8 and 13, preferably 9.5 and 11.5, which can be effected, for example, by adding base.

Useful bases are in particular alkali metal hydroxides, carbonates or hydrogencarbonates, although preference is given to sodium hydroxide and potassium hydroxide. The base can be used, for example, in solid form or in the form of aqueous solutions.

Subsequently, in step d), the reaction mixture obtained in step c) is acidified.

Preference is given to acidifying to a pH of 3.5 or less, more preferably to 0 to 3.5, and most preferably to 1 to 2.5.

Examples of useful acidifiers include acids or acidic salts having a pKa of 3.5 or less, preferably 0 or less. Particular preference is given to sulphuric acid or hydrochloric acid.

In a preferred embodiment of step d), initial acidification is effected only to a pH of above 3.5 and below 8, and undesired, soluble constituents are extractively removed as described above. Subsequently, the reaction mixture may then, for example, be freed of discolorations using a suitable adsorbent. Examples of useful adsorbents include silica gels, aluminium oxides, cellulose or activated carbon.

Subsequently, the mixture is further acidified.

The temperature on acidifying is not critical, although it may be advantageous to heat the reaction mixture to boiling, in order to drive out dissolved gases or decompose any sulphites present. The acidification protonates the salts of the compounds of the formula (I) and at least partly converts them to the free acids of the formula (I).

Preference is given to using acids having a pKa value in water of 3 or less in step d), particular preference to hydrochloric acid or sulphuric acid, and even greater preference to sulphuric acid.

The compounds of the formula (I) may be obtained in a manner known per se from the reaction mixtures obtained in step d), for example by extraction with organic solvent, filtration, centrifugation or sedimentation and decanting.

Examples of preferred solvents for the extraction are esters such as ethyl acetate and butyl acetate, aliphatic or aromatic, optionally halogenated hydrocarbons, for example petroleum, benzene, toluene, xylenes, chlorobenzene, dichlorobenzenes, isopropylbenzene, petroleum ether, hexane, heptane, octane, isooctane, cyclo-hexane, methylcyclohexane, dichloromethane, chloroform or carbon tetrachloride; ethers such as diethyl ether, methyl tert-butyl ether or diisopropyl ether, ketones such as 2-butanone or methyl isobutyl ketone or mixtures of such solvents.

The compounds of the formula (I) may be obtained after extraction in a manner known per se, for example by evaporating the solvent.

When the compounds of the formula (I) are removed by filtration, centrifugation or sedimentation and decanting, this may be effected, for example and with preference, at 0 to 70° C., more preferably 20 to 55° C.

For further purification, the compounds of the formula (I) may optionally be recrystallized or reprecipitated, although this is unnecessary.

The compounds of the formula (I) prepared by the process according to the invention, in particular 3-methoxy-2-methylbenzoic acid, are particularly suitable for use in a process for preparing pharmaceuticals and agrochemicals, for example crop protection agents and insecticides, or intermediates thereof, in particular acid chlorides, acid bromides, acid hydrazides, esters, for example 3-methoxy-2-methylbenzoyl chloride or bromide, 3-methoxy-2-methylbenzoic hydrazide, methyl 3-methoxy-2-methylbenzoate.

The advantage of the processes according to the invention is the efficient preparation of compounds of the formula (I), which enables them to be carried out in high yields without costly and inconvenient intermediate isolation.

### EXAMPLES

### Example 1

In a nickel autoclave, 300.0 g of trisodium 1,3,5-naphthalenetrisulphonate (70.9%, determined as the free acid) are stirred into a mixture of 232.0 g of aqueous sodium hydroxide solution (45%) and 194.0 g of sodium hydroxide in such a way that a readily stirrable, pasty suspension is obtained. The autoclave is closed and heated without stirring to 190° C. and the mixture is heated at this temperature for 30 minutes. Afterwards, the stirrer is switched on and the mixture is heated to 280° C. The internal pressure rises to 18.4 bar. The mixture is stirred at 280° C. for 6 hours and afterwards cooled to 90° C. At this temperature, 200.0 g of water are pumped in and the mixture is subsequently cooled to room temperature. The reaction mixture obtained in this way is filtered with suction to remove insolubles and washed with 187.3 g of water. 251.8 g of a brownish-white, finely divided solid and 795.7 g of a dark brown solution are obtained.

### Example 2

A flask is initially charged with 390.0 g of the solution from Example 1 and adjusted to a pH of 10.3 using 88.4 g of sulphuric acid (100%), and the reaction mixture is heated to 40 to 45° C. Dimethyl sulphate and sodium hydroxide solution are then simultaneously metered in at the same temperature in such a way that the pH remains in the range from 10.4 to 10.6. A total of 147.5 g of dimethyl sulphate and 85.8 g of sodium hydroxide solution (30%) are metered in within 2 hours. Afterwards, stirring is continued at the same temperature for 1 hour. On completion of the continuous stirring time, the pH is increased to 11 by metering in sodium hydroxide solution and the mixture is heated to 90° C. Stirring is continued at this temperature for 2 hours, and it is necessary to meter in further sodium hydroxide solution (30%). The reaction solution is cooled to 70° C. 20.0 g of Acticarbon F® activated carbon are added and a pH of 6.5 is set by metering in 32.9 g of sulphuric acid (100%). The mixture is heated to reflux, and the reaction solution is clarified and washed with 550.0 g of water. The clarified reaction solution is returned to the reactor, heated to 90° C. and set to a pH of 3 by metering in 18.9 g of sulphuric acid (100%). The mixture is heated at 90° C. for 1 hour. A pH of 3 is then set using 41.6 g of sulphuric acid (100%). The mixture is heated at 90° C. for 1 hour. A pH of 1 is then set using 41.6 g of sulphuric acid (100%) and the reaction mixture is cooled to 45° C. within 4 hours. The product precipitates out as a white precipitate. The product is filtered off with suction and washed with 300.0 g of water. The precipitate is dried in a vacuum drying cabinet. 1 532.9 g of washing/mother liquor (0.03% of methoxymethylbenzoic acid) and 29.3 g of 3-methoxy-2-methylbenzoic acid (purity 95.9%) are obtained. This corresponds to an isolated yield based on trisodium 1,3,5-naphthalenetrisulphonate of 60% of theory.

### Example 3

In a nickel autoclave, 300.0 g of trisodium 1,3,5-naphthalenetrisulphonate (70.9%, determined as the free acid) are stirred into a mixture of 356.0 g of aqueous sodium hydroxide solution (45%) and 126.0 g of sodium hydroxide pastilles and 127.0 g of calcium hydroxide in such a way that a readily stirrable, pasty suspension is obtained. The autoclave is closed and heated without stirring to 190° C. and the mixture is heated at this temperature for 30 minutes. Afterwards, the stirrer is switched on and the mixture is heated to 280° C. The internal pressure (autogenous pressure) rises to 29.6 bar. The mixture is stirred at the reaction temperature for 15 hours and afterwards cooled to 90° C. At this temperature, 200.0 g of water are pumped in and the mixture is subsequently cooled to room temperature. The suspension obtained is filtered off with suction and washed with 695.5 g of water. 299.6 g of a brownish-white, finely divided solid and 1 287.5 g of a dark brown solution are obtained.

### Example 4

A flask is initially charged with 300.0 g of the solution from Example 3 which are diluted with 50.0 g of water. A pH of 10.3 is set using 42.8 g of sulphuric acid (100%) and the reaction mixture is heated to 40 to 45° C. Dimethyl sulphate and sodium hydroxide solution are then simultaneously metered in at 40 to 45° C. in such a way that the pH remains in the range from 10.4 to 10.6. 65.1 g of dimethyl sulphate and 45.0 g of sodium hydroxide solution (30%) are metered in within 2 hours. Afterwards, stirring is continued at the same temperature for 1 hour. On completion of the continuous stirring time, the pH is increased to 11 by metering in sodium hydroxide solution and the mixture is heated to 90° C. Stirring is continued at this temperature for 2 hours, and it is necessary to meter in further sodium hydroxide solution (30%). The reaction solution is cooled to 70° C. 3.0 g of activated carbon are added and a pH of 7.8 is set by metering in 13.1 g of sulphuric acid (100%). The mixture is heated to reflux, and the reaction solution is clarified using activated carbon and washed with 250 g of water. The clarified reaction solution is returned to the reactor, heated to 90° C. and set to a pH of 4 by metering in 11.4 g of sulphuric acid (100%). The mixture is heated at 90° C. for 1 hour. A pH of 1 is then set using 38.4 g of sulphuric acid (100%) and the reaction mixture is cooled to 45° C. within 4 hours. The product precipitates out as a white precipitate. The product is filtered off with suction on a glass suction filter and washed with 250 g of water. The precipitate is dried in a vacuum drying cabinet. 784.7 g of mother liquor (0.02% of methoxymethylbenzoic acid), 238.0 g of washing liquor (methoxymethylbenzoic acid not detectable) and 16.9 g of 3-methoxy-2-methylbenzoic acid (purity 95.5%) are obtained. This corresponds to an isolated yield based on trisodium 1,3,5-naphthalenetrisulphonate of 72.1% of theory.

### Example 5

### (Methyl 3-methoxy-2-methylbenzoate)

In a flat-flanged vessel, 200.0 g of 3-hydroxy-2-methylbenzoic acid (purity 96.9%) are dissolved in a mixture of 200.0 g of water and 173.3 g of sodium hydroxide solution (w=30%):

This results in a pH of approx. 5.5. The clear solution is heated to 40° C. and set to pH=10.5 by metering in sodium hydroxide solution (w=30%). Dimethyl sulphate and sodium hydroxide solution are now metered in simultaneously at 40-45° C. in such a way that the pH remains within the range of 10.4-10.6. 401.5 g of dimethyl sulphate and 147.9 g of sodium hydroxide solution (w=30%) are metered in within 2 h. Afterwards, the mixture is stirred at 40-45° C. for a further 2 h. Afterwards, the aqueous phase is removed and discarded. The organic phase is washed with 200.0 g of water. The washing water is removed and discarded. 223.56 g of crude methyl 3-methoxy-2-methylbenzoate remain.

For further purification, the crude product is subjected to a vacuum distillation through a Vigreux column. At a vacuum of 20 mbar, 5.7 g of first runnings and 205.5 g of main fraction (top temperature 145° C., methyl 3-methoxy-2-methylbenzoate, purity 98.7%) distil over.

205.0 g of methyl 3-methoxy-2-methylbenzoate, purity 98.7% of MMBE and 0.00% of 3-hydroxy-2-methylbenzoic acid, methyl 3-hydroxy-2-methylbenzoate, 3-methoxy-2-methylbenzoic acid, corresponds to 88.5% of theory (based on 3-hydroxy-2-methylbenzoic acid).

### Example 6

### (3-Methoxy-2-methylbenzoic acid)

In a flat-flanged vessel, 213.1 g of 3-hydroxy-2-methylbenzoic acid (purity 92.75%) are dissolved in a mixture of 200.0 g of water and 173.3 g of sodium hydroxide solution (w=30%):

This results in a pH of approx. 5.5. The clear, brown solution is heated to 40° C. and set to pH=10.5 by metering in sodium hydroxide solution (w=30%). Dimethyl sulphate and sodium hydroxide solution are now metered in simultaneously at 40-45° C. in such a way that the pH remains within the range of 10.4-10.6. 401.5 g of dimethyl sulphate and 127.2 g of sodium hydroxide solution (w=30%) are metered in within 2 h. Afterwards, the mixture is stirred at 40-45° C. for a further 2 h. Afterwards, the aqueous phase is removed and discarded. 250.0 g of water are added to the organic phase and the mixture is heated to 90° C. The mixture is stirred at this temperature for a further 2 h and the pH is maintained at 10.5-11 by metering in sodium hydroxide solution (w=30%). The organic phase disappears and a solution forms. The reaction solution is cooled to 80° C.

A second flat-flanged vessel is charged with 250.0 g of water and heated to 80° C. The reaction mixture of the first vessel and sulphuric acid (w=100%) are simultaneously metered in at 80° C. in such a way that a pH of 3.8-4.2 is maintained. The product precipitates out as a white precipitate. After metering in approx. 10% of the reaction solution the mixture is heated for approx. 1 h. On completion of metering in, the pH is adjusted to 1 using sulphuric acid (w=100%) and the mixture is cooled to 40° C. within 4 h. The product is filtered off with suction and washed with 2×500.0 g of water. The precipitate is dried in a vacuum drying cabinet at 60° C. for 16 h. 202.0 g of 3-methoxy-2-methylbenzoic acid, purity 96.6% of MMBA (0.00% of 3-hydroxy-2-methylbenzoic acid, methyl 3-hydroxy-2-methylbenzoate, methyl 3-methoxy-2-methylbenzoate) are obtained, corresponding to 90.4% of theory (based on 3-hydroxy-2-methylbenzoic acid).

### Example 7

### (3-Methoxy-2-methylbenzoyl chloride)

A three-necked flask is initially charged with 136.0 g of thionyl chloride under nitrogen and heated to 60° C. A melt at 180° C. of 136.0 g of 3-methoxy-2-methylbenzoic acid is added dropwise within one hour. The reaction commences immediately with evolution of gas (SO₂, HCl). After the addition is complete, the mixture is heated to reflux (80° C.) over one hour and stirred at this temperature until the gas evolution subsides (approx. 2 h). The solution is cooled to approx. 50° C. under nitrogen and the reflux condenser is replaced by a Vigreux column. 14.0 g of thionyl chloride are distilled off at atmospheric pressure (liquid phase up to 170° C.) and the remaining liquid phase is fractionated under reduced pressure (10 mbar). The distillate obtained is 139.4 g of 3-methoxy-2-methylbenzoyl chloride (98.8%). This corresponds to 94.2% of theory.

### Example 8

### (3-Methoxy-2-methylbenzoyl chloride)

In a flask, 91.9 g of 3-methoxy-2-methylbenzoic acid are suspended under nitrogen in 200.0 g of dry xylene. 119.0 g of thionyl chloride are metered in at room temperature. The grey suspension is heated to 80° C. within one hour, and the reaction commences at approx. 30° C. with gas evolution (SO₂, HCl), resulting in a homogeneous solution at about 50° C. Afterwards, the reaction temperature is raised to 135° C. within a further hour. At this temperature, stirring is continued to completion of gas evolution (1 h). Afterwards, the reflux condenser is replaced by a small Vigreux column with a distillation bridge, and first the excess approx. 45.0 g of thionyl chloride and afterwards, at a maximum bottom temperature of 154° C., the majority of the xylene (approx. 165 g) is distilled off. Assuming quantitative conversion, 54.4% of the remaining 180.0 g of bottoms consists of 3-methoxy-2-methylbenzoyl chloride. This corresponds to 0.53 mol or 97.8 g of 100% 3-methoxy-2-methylbenzoyl chloride. The bottoms are liquid at temperatures of approx. 30° C. and are used directly for the further reaction.

### Example 9

### (3-Methoxy-2-methylbenzoic tert-butylhydrazide)

In a flat-flanged vessel, 81.3 g of tert-butylhydrazine hydrochloride are dissolved in 230.0 g of water. A pH of 9.2 is set by metering in 55.8 g of sodium hydroxide solution (w=30%). 150.0 g of 80/110 special-boiling-point gasoline (boiling range 80-110° C.) are added and the mixture is cooled to 15° C. The xylenic 3-methoxy-2-methylbenzoyl chloride solution prepared under Example 1 and sodium hydroxide solution are now simultaneously metered in at 15-20° C. in such a way that the pH remains within the range of 9.0-9.5. 180.0 g of xylenic MMBC solution (w=30%) and 97.3 g of sodium hydroxide solution (w=30%) are metered in within 1.5 h. The pump and the feeds are flushed with 43.0 g of xylene, and this xylene is added to the reaction mixture. On completion of metering, stirring is continued at 10-15° C. for another hour. Afterwards, the reaction mixture is heated to 40° C. within 45 min and stirred at this temperature for 2 h to complete the reaction.

The suspension is heated to 70° C., and the solid dissolves completely in the organic phase. The aqueous phase is removed and discarded. The organic phase is washed with 200.0 g of demineralized water, and the washing water is likewise discarded.

2.0 g of activated carbon are added to the organic phase, and the mixture is heated to 80° C. and filtered through a filter paper. The filter cake is washed with 43.0 g of xylene, and the filtrates are combined and processed further.

300.0 g of 80/110 special-boiling-point gasoline are added to the clarified organic phase at 60-80° C. and the mixture is cooled to 5° C. within 6 h. At approx. 38-40° C., the product begins to precipitate out. The precipitated product is filtered off with suction and washed with 80/110 special-boiling-point gasoline. The precipitate is dried at 60° C. and 200 mbar for 20 h in a vacuum drying cabinet. 109.2 g of 3-methoxy-2-methylbenzoic tert-butylhydrazide (purity 95.4%, one further component visible in HPLC) are obtained. This corresponds to a yield based on 3-methoxy-2-methylbenzoic acid of 83.2% of theory.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing compounds of the formula (I) 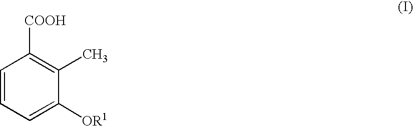
where
R¹ is C₁-C₁₄-alkyl, C₇-C₂₀-arylalkyl, C₁₃-C₂₀-diarylalkyl or radicals of the formulae (IIa) or (IIb)
A-OR² (IIa)
A-NR³R⁴ (IIb)
where
A is in each case a C₁-C₄-alkylene radical and R² and also R³ and R⁴ are each independently methyl, ethyl and isopropyl,
comprising reacting
a) compounds of the formula (III) 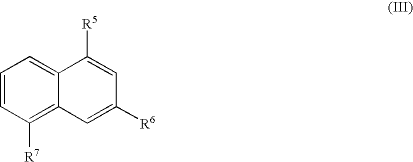
where
R⁵, R⁶ and R⁷ are each independently hydrogen, hydroxyl, amino or SO₃M where M is hydrogen, ammonium, an alkali metal or half of an equivalent of an alkaline earth metal,
with alkali metal hydroxide in the presence of water and
b) partially neutralizing the reaction mixure obtained in step a),
c) reacting the reaction mixtures obtained in step b) with compounds of the formulae (IVa), (IVb) or (IVc)
R¹—X (IVa)
R¹—OSO₂—R⁸ (IVb)
R¹—OSO₂—OR¹ (IVc)
where
R¹ is as defined above and
X is chlorine, bromine or iodine and
R⁸ is C₁-C₄-alkyl, C₁-C₄-perfluoroalkyl, phenyl or p-tosyl and
c) acidifying the reaction mixtures obtained in step c).

2. Process according to claim 1, characterized in that step a) is carried out in the presence of alkaline earth metal hydroxide.

3. Process according to claim 1, characterized in that step a) is carried out in the presence of calcium hydroxide.

4. Process according to claim 1, characterized in that, in step a), 3-amino-1,5-naphthalenesulphonic acid, its mono- or dialkali metal salts, 1,3,5-naphthalenetrisulphonic acid or its mono-, di- or trialkali metal salts are used.

5. Process according to claim 1, characterized in that, in step a), trialkali metal salts of 1,3,5-naphthalenetrisulphonic acid are used.

6. Process according to claim 1, characterized in that, in step a), sodium hydroxide or potassium hydroxide or a mixture thereof is used as the alkali metal hydroxide.

7. Process according to claim 1, characterized in that, in step b), insoluble constituents are removed.

8. Process according to claim 1, characterized in that, in step b), the pH is adjusted to 8 to 13.

9. Process according to claim 1, characterized in that, in step c), methyl chloride, methyl iodide, methyl mesylate, methyl p-tosylate or dimethyl sulphate are used.

10. Process according to claim 1, characterized in that, in step c), the pH is maintained between 8 and 13 in the course of the reaction.

11. Process according to claim 1, characterized in that, in step d), initial acidification is effected to a pH above 3.5 and below 8 and undesired, soluble constituents are extractively removed.

12. Process for preparing pharmaceuticals and agrochemicals comprising providing therefor compounds of the formula (I) which have been prepared by a process according to claim 1.
